(19) United States
(12) Patent Application Publication
(10) Pub. No.: US 2023/0301893 A1
(43) Pub. Date: Sep. 28, 2023

(54) Hair Growth and Scalp Preparations

(71) Applicant: Meng Teng LIM, Island East (HK)

(72) Inventors: Chanda Zaveri, Rancho Palos Verdes, CA (US); Meng Teng LIM, Island East (HK)

(21) Appl. No.: 17/802,038

(22) PCT Filed: Feb. 28, 2020
(86) PCT No.: PCT/US2020/020527

§ 371 Date: Aug. 24, 2022

(51) Int. Cl. A61K 8/64; A61K 8/23; A61K 8/44; A61K 8/9789; A61Q 7/00; A61K 8/49

(52) U.S. Cl. CPC A61K 8/64; A61K 8/23; A61K 8/447; A61K 8/4946; A61K 8/9789; A61Q 7/00

(57) ABSTRACT

Preparations and methods disclosed herein may be used for transdermal delivery of a peptide comprising a sequence of L-K-E-K-K (SEQ ID NO: 1) to the scalp for hair growth stimulation. In some embodiments, preparations and methods disclosed herein may be used for transdermal delivery of the peptide comprising SEQ ID NO: 1 along with bacterially and/or microbially active ingredients.

### CROSS-REFERENCE TO RELATED APPLICATIONS

This application is a 371 national stage application of international application No. PCT/US2020/020527, filed Feb. 28, 2020, which is hereby incorporated by reference in its entirety.

### INCORPORATION OF SEQUENCE LISTING

This document incorporates by reference herein an electronic sequence listing text tile, which was filed in electronic format via EFS-Web on Jun. 2, 2023. The text file is named “1-20US_Seq_Listing2_ST25.txt,” is 1 kilobyte, and was created on Apr. 16, 2020.

### BACKGROUND

Each hair is composed of two distinct structures: the dynamic hair follicle located in the dermis and the hair shaft, a hard keratinized part that extends above the skin surface. Hair grows in cycles delimitated by three distinct phases: anagen, catagen, and telogen. Anagen is the growth phase during which new materials are deposited in the hair shaft by rapidly dividing follicular cells. Anagen scalp hair grows by 1 cm per month for a period of 2-6 years. The duration of the anagen period dictates the maximal length of hair and is genetically determined. Catagen is a transition phase, lasting for about 2-3 weeks, marked by a stop of hair growth. During this phase, the hair follicle involutes, becomes attached to the hair shaft and keratinizes forming a club hair that is pushed upward toward the scalp, as the dermal papilla breaks away. Telogen is the resting phase. The hair follicle regresses, becomes fully keratinized, and can easily be pulled out. The telogen phase lasts around 3 months for scalp hair. Following shedding, the next hair can start growing as the papilla and the follicle join again. An adult healthy scalp normally bears 70-85% hair in the anagen phase and 10-15% in the telogen phase, the rest being in the catagen phase. Hair loss is generally associated with a shortening of the anagen phase and premature entry into the catagen phase. During normal hair physiology, hair cycles through anagen (growth phase), catagen (transition phase), and telogen (resting and falling phase) before reentering early anagen to initiate the growth of a new hair.

Hair loss and baldness (alopecia) are common phenomena in mammals, including humans, where it is extremely common in adult males, and also occurs in adult females. In fact, some degree of alopecia on the vertex from puberty onwards is thought to be a universal phenomenon in both men and women.

Hair loss may be naturally occurring (primary alopecia) or it may be induced by chemical or physical agents (secondary alopecia). Alopecia is also frequently observed in both pre- and post-pubertal patients as a side effect of anticancer chemotherapy. Hair loss may also result from specific disease states, such as mange, or formation of scar tissue from bites and with increasing age.

The physical phenomenon of hair loss may lead to psychological problems in the patient, decreased social activity, and the development of psychological diseases. In the case of cancer patients, the likelihood of chemotherapy-induced alopecia may lead to a refusal to accept treatment. As a result of the prevalence of alopecia, and its potentially devastating impact, there is immense interest in the development of effective clinical treatments, both to prevent hair loss and to stimulate regrowth of lost hair.

Despite the widespread occurrence of alopecia, the need for prevention and therapy, and extensive research efforts to find suitable remedies, there remains an urgent need for effective treatment. For example, lack of a proven and effective treatment for alopecia has caused many afflicted individuals to adopt the practice of wearing a wig or toupee. Another extreme measure used to combat alopecia, hair transplant surgery, is not available as an option in many cases, e.g., following chemotherapy, and offers, at best, only a partial remedy.

A common non-surgical treatment for stimulating hair growth, which is currently used clinically, is minoxidil (The Upjohn Company, Kalamazoo, Mich.). A solution containing minoxidil as an active ingredient is known as Rogain®. As stated in the Rogaine® Patient Information Booklet (The Upjohn Company, Kalamazoo., Mich., revised June 1992), minoxidil is a vasodilatory drug which has serious side effects when administered orally for the treatment of hypertension. At the same time, topical application of minoxidil for the treatment of alopecia is only partially effective and suffers from a number of disadvantages. For example, it is only recommended for treatment of male pattern alopecia of the vertex (or frontal recession), has to be applied twice daily for at least four months, and requires a normal scalp with no local abrasions, dermatitis or sunburn - conditions that can increase absorption into the blood stream and the concomitant risk of side effects. Further, minoxidil is of limited effectiveness insofar as for those patients who do respond to minoxidil treatment, the new hair is likely to be shed within a few months after stopping treatment.

Moreover, it is recognized that delivering such active ingredients through the skin, i.e., transdermally, as opposed to other methods of parenteral administration is extremely difficult. In this regard, in order to be effective, an active agent for hair treatment must pass through the outer layer of skin or epidermis and into the dermis layer before being absorbed into the bloodstream. The epidermis comprises two main parts, the stratum corneum and the stratum germinativum. The stratum corneum forms the outermost layer of the epidermis and consists of many stratified layers of compacted, flattened, keratinized cells that have lost their nuclei. This outermost layer serves as a physical barrier to microorganisms and also to chemical agents. In particular, it behaves as a primary barrier to percutaneous absorption of drugs. Because of the barrier effect of the skin, it has generally only been possible to deliver drugs that are “low-dose” drugs, i.e., in the range of 15 mg/day or less, or those of low molecular weight. In addition, drugs for transdermal delivery must have the proper lipophilic-hydrophilic balance to permit adequate absorption, with lipid-soluble substances having comparatively greater skin permeability than water-soluble substances.

Recently, an orally-administered systemic agent, namely, finasteride, sold under the trademark Propecia® produced by Merck and Company of West Point, Penn., has proven clinically effective in treating alopecia in men with male pattern hair loss (androgenetic alopecia). Propecia® is a competitive and specific inhibitor of Type II 5α-reductase, an intracellular enzyme that converts androgen testosterone into dihydrotestoterone (DHT). Administration of Propecia® decreases scalp and serum DHT concentrations and, by this mechanism, appears to interrupt the enzymatic pathway attributable to the development of androgenetic alopecia in those patients genetically predisposed to such condition.

While clinically effective in treating male pattern hair loss, Propecia® is known to produce significant adverse reactions. These adverse reactions include sexual dysfunction, as well as reported incidences of breast tenderness and enlargement. This composition is further extremely teratogenic and must not be handled by pregnant women insofar as Propecia® is suspected of causing impaired sexual organ development in male fetuses.

Accordingly, there is substantial need in the art for compositions that are effective at stimulating hair growth and capable of transdermal delivery of hair growth promoters while being safe, easy to apply, and relatively inexpensive when compared to other hair loss treatments.

### SUMMARY

A substantially purified peptide having physiological activity associated with wound healing was disclosed in U.S. Pat. No. 6,767,891, which is incorporated herein by reference in its entirety. The peptide disclosed in the ‘891 patent has a sequence comprising L-K-E-K-K (SEQ ID NO: 1), wherein the peptide is in linear or cyclic form, and wherein when the peptide is in linear form, the amino terminus is optionally acetylated.

The preparations and methods disclosed herein may be used for transdermal delivery of the peptide comprising SEQ ID NO: 1 to the scalp for hair growth stimulation and rejuvenation.

Additionally, many patients suffer simultaneously from hair loss, scalp or follicle bacteria, and/or microbial infections. In some cases, bacteria may contribute to hair loss, and mites, such as Demodex mites, may spread bacteria, thereby exacerbating bacterial problems. When a bacterial or microbial problem is deeply rooted at the hair follicle, transdermal delivery of bacterial or microbial agents is important. Therefore, in some embodiments, preparations and methods disclosed herein may be used for transdermal delivery of the peptide comprising SEQ ID NO: 1 along with bacterially and/or microbially active ingredients.

In an aspect, a preparation comprises a peptide comprising a sequence of L-K-E-K-K (SEQ ID NO: 1) and caprylyl glycol.

In an embodiment, the peptide comprises at least 5 amino acids. In an embodiment, the peptide has a weight average molecular weight of 150 Daltons to 200 Daltons.

In an embodiment, the peptide is present at a concentration between 0.1 mg and 0.5 mg and the caprylyl glycol is present at a concentration between 1 gram and 5 grams.

In an embodiment, the preparation further comprises colloidal sulfur, which may be present at a concentration between 1 gram and 3 grams.

In an embodiment, the preparation further comprises a sulfured amino acid, which may be present at a concentration between 1 mg and 5 mg. In an embodiment, the sulfured amino acid is selected from the group consisting of methionine, cysteine, homocysteine and taurine.

In an embodiment, the preparation further comprisies a sulfo mucopolysaccharide extract, which may be present at a concentration between 0.5 gram and 1 gram. In an embodiment, the sulfo mucopolysaccharide is selected from the group consisting of chondroitin sulfate, dermatan sulfate, keratin sulfate, heparin, heparin sulfate, and hyaluronan (hyaluronic acid).

In an embodiment, the preparation further comprises trifolium pratense flower extract, azadirachta indica extract, eclipta prostrata extract or a combination thereof. In an embodiment, oenothera biennis (evening primrose) extract comprises:
linoleic acid 73.88 ± 0.09
γ-linolenic acid 9.24 ± 0.05
oleic acid 6.93 ± 0.02
palmitic acid 6.31 ± 0.14
stearic acid 1.88 ± 0.02
vaccenic acid 0.81 ± 0.03
eicosenoic acid 0.55 ± 0.01
eicosanoic acid 0.31 ± 0.03
behenic acid 0.10 ± 0.01

In an embodiment, the preparation further comprises a compound of formula (I):

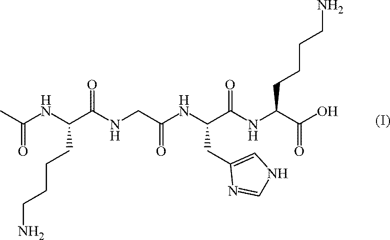 [SMILES: CC(=O)N[C@@H](CCCCN)C(=O)NCC(=O)N[C@@H](Cc1c[nH]cn1)C(=O)N[C@@H](CCCCN)C(=O)O]

In an embodiment, the preparation further comprises one or more carriers, excipients, preservatives, fragrances and/or diluents.

In an aspect, an antibacterial shampoo includes a preparation comprising a peptide comprising a sequence of L-K-E-K-K (SEQ ID NO: 1) and caprylyl glycol. In an embodiment, the antibacterial shampoo is a follicle antibacterial shampoo, i.e., a shampoo having an antibacterial active ingredient and a delivery agent capable of increasing the concentration of antibacterial agent that reaches a subject’s hair follicles relative to the amount that would reach the subject’s hair follicles in the absence of the delivery agent. For example, the delivery agent may be an ingredient capable of transdermal transport. In an embodiment, the delivery agent is chemically or physically bound to or associated with the antibacterial agent. In an embodiment, the delivery agent is not chemically or physically bound to or associated with the antibacterial agent, but the delivery agent opens the dermal structure, which allows other agents to penetrate the dermis.

In an aspect, a hair growth product includes a preparation comprising a peptide comprising a sequence of L-K-E-K-K (SEQ ID NO: 1) and caprylyl glycol.

In an aspect, a method of reducing a concentration of bacteria from the hair or scalp of a subject in need thereof comprises administering to the subject a therapeutically effective amount of a preparation disclosed herein.

In an aspect, a method of reducing a concentration of microbes from the hair or scalp of a subject in need thereof comprises administering to the subject a therapeutically effective amount of a preparation disclosed herein.

In an aspect, a method of inducing hair growth in a subject in need thereof comprises administering to the subject a therapeutically effective amount of a preparation disclosed herein.

In an embodiment, the preparation is administered topically. In an embodiment, the preparation is administered at least once daily.

### BRIEF DESCRIPTION OF THE DRAWINGS

FIG. 1 is a graph showing the effects of the peptides TB₄ and TA₁ on endothelial cell migration in a Boyden chamber migration assay.

FIG. 2 is a graph showing the effects of the peptides TB₄ and TA₁ on proliferation of endothelial cells at 4 hours and 24 hours after stimulation as measured in a MTT (tetrazolium) assay.

### DETAILED DESCRIPTION

In general, the terms and phrases used herein have their art-recognized meaning, which can be found by reference to standard texts, journal references and contexts known to those skilled in the art. The following definitions are provided to clarify their specific use in the context of this description.

An “amino acid” is a molecular building block of protein. An “amino acid residue” is the simplest discreet unit or monomer of a protein chain or peptide.

In the context of this specification, the term “substantially purified” refers to a state of purity that is at least 50%, preferably at least 70%, more preferably at least 85%, and still more preferably at least 95%, and in which the peptide having physiological activity is present in the substantial absence of other peptides or proteins having physiological activity.

“Sulfo mucopolysaccharides” are also called glycosaminoglycans (GAGs). They are a family of highly sulfated, complex, polydisperse linear polysaccharides.

### Peptide TA₁

One of the peptides comprising SEQ ID NO: 1 is a 28-amino acid peptide designated TA₁. The peptide has the sequence SEQ ID NO: 2: Ac-S-D-A-A-V-D-T-S-S-E-I-T-T-K-D-L-K-E-K-K-E-V-V-E-E-A-E-N. In this peptide and other peptides disclosed herein, the notation “Ac” at the amino terminus of the peptide indicates that the amino terminus is acetylated. Generally, this acetyl group can be cleaved without impairing the function of the peptide. This peptide is linear. This peptide has an acetylated amino terminus and has a molecular weight of 3071 Daltons. The isoelectric point of this peptide is 4.1, indicating a predominance of acidic amino acids. This peptide may be derived by cleavage of thymosin.

### Peptide TB₄

Peptide TB₄ is a peptide of 44 amino acids. This peptide has the sequence SEQ ID NO: 3: Ac-A-N-K-G-Q-A-P-G-E-A-M-K-P-S-F-L-K-E-K-K-E-V-V-E-R-S-K-E-E-E-G-P-A-K-M-N-L-V-I-E-M-P-K-D. This peptide is linear. The amino terminus of this peptide is acetylated. This peptide contains the conserved sequence L-K-E-K-K (SEQ ID NO: 1).

Peptides having conservative amino acid substitutions except in the highly conserved sequences of L-K-E-K-K (SEQ ID NO: 1) are within the scope of the present invention. It is a well-established principle of protein and peptide chemistry that certain amino acid substitutions, called “conservative” amino acid substitutions, can frequently be made in a protein or a peptide without altering either the conformation or the function of the protein or peptide. Such changes include substituting any of isoleucine (I), valine (V), and leucine (L) for any other of these amino acids; aspartic acid (D) for glutamic acid (E) and vice versa; glutamine (Q) for asparagine (N) and vice versa; and serine (S) for threonine (T) and vice versa. The above-mentioned substitutions are not the only amino acid substitutions that can be considered “conservative”. Other substitutions can also be considered conservative, depending on the environment of the particular amino acid. For example, glycine (G) and alanine (A) can frequently be interchangeable, as can be alanine (A) and valine (V). Methionine (M), which is relatively hydrophobic, can frequently be interchanged with leucine (L) and isoleucine (I), and sometimes with valine (V). Lysine (K) and arginine (R) are frequently interchangeable in locations in which the significant feature of the amino acid residue is its charge and the differing pK’s of these two amino acid residues are not significant. Cysteine (C) can frequently be replaced by serine (S) when cysteine’s capacity to form disulfide bonds is either undesirable or unneeded. Still other changes can be considered “conservative” in particular environments.

As indicated above, the peptides disclosed herein can be either circular or linear. The specific peptides described above, however, are linear. Some of the peptides have their amino termini blocked, typically by acetylation. However, these acetyl groups can be cleaved by hydrolysis without interfering with the function of the peptides.

### Nucleic Acids Encoding the Peptides

Peptides disclosed herein may be encoded by isolated nucleic acids. As used herein, the term “nucleic acid” includes both DNA and RNA and both single-stranded and double-stranded forms; if double-stranded, DNA-RNA hybrids are also included. Recitation of a single-stranded nucleic acid sequence also includes its complement according to the generally accepted Watson-Crick rules for base pairing. Nucleic acids encoding these peptides can either be DNA or RNA; however, in many applications, DNA is preferred.

The term “isolated” is used herein to indicate that the nucleic acids are present in substantial isolation from nucleic acid molecules that do not encode a peptide disclosed herein. In the context of this specification, the term “isolated” refers to a state of purity that is at least 50%, preferably at least 70%, more preferably at least 85%, and still more preferably at least 95%.

However, nucleic acids can be incorporated into larger nucleic acid molecules such as vectors for transfection of appropriate host cells and production of a peptide, and the term “isolated” is not to be interpreted to preclude this incorporation into larger, genetically-engineered molecules not occurring in nature.

The sequence of the nucleic acids is chosen according to the conventional triplet genetic code to encode the amino acid sequence of the particular peptides. Because the genetic code, which specifies amino acids by triplet codons in the nucleic acid sequence, is degenerate, and many amino acids are specified by more than one codon, all possible alternatives of codons can be used. However, in some cases, the efficiency of transcription and/or translation of the nucleic acid sequences can be affected by the codon selection. In such cases, it is preferred to use codons that provide increased efficiency of transcription and/or translation of the nucleic acid sequences.

### Vectors and Host Cells

A vector comprising a DNA operably linked to at least one control element that influences the expression of the DNA is also contemplated. These control elements can be promoters, operators, enhancers, or other nucleic acid sequences that affect the expression of the DNA. The vector can be derived from either prokaryotic or eukaryotic sources. The vector can comprise sequences of chromosomal, non-chromosomal, or synthetic DNA sequences. Typically, these vectors include one or more cloning sites that contain restriction endonuclease sequences that are readily cleavable by specific restriction endonucleases. It is generally preferred that these restriction endonucleases yield cohesive or “sticky” ends for more efficient cloning of the desired sequence. Some suitable prokaryotic cloning vectors include plasmids from Escherichia coli, such as colE1, pCR1, pBR322, pMB9, pUC, pKSM, or RP4. Prokaryotic vectors also include derivatives of bacteriophage DNA such as M13 and other filamentous single-stranded DNA phages. Other vectors, such as baculovirus vectors, can be used.

Examples of useful expression controlled sequences are the lac system, the trp system, the tac system, the trc system, major operator and promoter regions of bacteriophage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters of SV40 and other sequences known to control the expression of genes of prokaryotic or eukaryotic cells and their viruses or combinations thereof. Vectors useful in yeast are available. A suitable example is the 2µ plasmid. Vectors for use in animal cells are also known. These vectors include derivatives of SV40, adenovirus, retrovirus-derived DNA sequences, and shuttle vectors derived from combinations of functional mammalian vectors, such as those described above, and functional plasmids and phage DNA. Another suitable vector is the baculovirus vector. In general, however, it is preferred to use a vector that is suitable for expression in E. coli.

Vectors are inserted into a host cell for expression. Typically, these vectors are inserted into a host cell by methods well known in the art, such as transfection, transformation, electroporation, direct injection of the DNA, lipofection, and other well-understood methods. The method to be used can be chosen according to the host cells selected and the size and conformation of the DNA. Some useful expression host cells include well-known prokaryotic and eukaryotic cells. Some suitable prokaryotic hosts include, for example, E. coli, such as E. coli SG-936, E. coliHB101, E. coli W3110, E. coli •1776, E coli •2282, E. coli DHI, and E. coli MRCI. Other bacterial and fungal host cells could be used, such as Pseudomonas, Bacillus species, such as Bacillus subtilis, and Streptomyces. Other host cells that can be used are eukaryotic cells such as yeast and other fungi, insect cells, animal cells, such as COS cells and CHO cells, human cells, and plant cells in tissue culture.

### Methods of Preparation of Peptides

### Solid-State Peptide Synthesis

Peptides can be synthesized by standard solid-state peptide synthesis methods, such as those described in M. Bodanszky, “Principles of Peptide Synthesis” (Springer-Verlag, Berlin, 2d ed., 1993). This involves synthesis on an insoluble polymer such as a styrene-divinylbenzene copolymer that is derivatized. The sequence of reactions used is standard.

### Genetic Engineering

Peptides can be prepared by genetic engineering. In general, a method of producing a substantially purified peptide having a physiological activity comprises the steps of: (1) culturing a host cell transfected with a vector comprising DNA encoding the peptide operably linked to at least one control element that influences the expression of the DNA; and (2) isolating the peptide produced by the host cell to produce the substantially purified peptide.

Expression methods are described in, e.g., D. V. Goeddel, “Gene Expression Technology” (Academic Press, San Diego, 1991). In general, such methods are well known in the art.

Once expressed, the peptides can be isolated by standard protein isolation techniques including ion-exchange chromatography on resins such as diethylaminoethylcellulose or carboxymethylcellulose, chromatography on size exclusion media (gel filtration), isoelectric focusing, chromatofocusing, and other standard methods, such as those described in R. K. Scopes, “Protein Purification: Principles and Practice” (3d Ed., Springer-Verlag, New York, 1994).

If polyclonal or monoclonal antibodies are prepared for these peptides, these antibodies can be used in affinity chromatography by standard methods such as those described in the above-identified Scopes book. Such methods for the preparation of polyclonal antibodies or monoclonal antibodies are well known in the art and need not be described in further detail here. In general, polyclonal antibodies are produced by injecting the peptides, with or without a suitable adjuvant such as Freund’s complete adjuvant, into an antibody-producing mammal such as a rat, a rabbit, a sheep, or a goat. The peptide can be coupled to a carrier protein such as keyhole limpet hemocyanin. Once polyclonal antibodies are produced, cells producing such polyclonal antibodies can be fused with appropriate fusion partners by standard techniques to yield hybridomas producing monoclonal antibodies of defined specificity.

### Methods of Use

Peptides can be administered by a number of routes. When used for stimulating hair growth, they are typically administered topically to the skin of the scalp as part of a preparation, which may be in the form of a cream, oil, shampoo, spray, tincture, ointment or the like.

A preferred dose is 0.5 ml of 100 µg/ml solution of the peptide or a dose of 50 µg of the peptide. The dosages to be administered can be determined by one of ordinary skill in the art depending on the clinical severity of the problem, the age and weight of the patient, the exposure of the patient to conditions that may affect the course of hair growth, the existence or nonexistence of underlying systemic problems such as diabetes, impaired circulation, and immunocompromised status, and other pharmacokinetic factors generally understood in the art, such as liver and kidney metabolism. The interrelationship of dosages for animals of various sizes and species and humans based on mg/m³ of surface area is described by E. J. Freireich et al., “Quantitative Comparison of Toxicity of Anticancer Agents in Mouse, Rat, Hamster, Dog, Monkey and Man,” Cancer Chemother. Rep. 50: 219-244 (1966). Adjustments in the dosage regimen can be made to optimize the therapeutic response. Doses can be divided and administered on a daily basis or the dose can be reduced proportionally depending on the therapeutic situation.

The active ingredient is often mixed with diluents or excipients that are physiologically tolerable and compatible with the active ingredient. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like. For a more detailed description of the foregoing see a standard pharmaceutical text such as Remington’s Pharmaceutical Sciences, Mack Publishing Co. Easton, Pa. (1970).

Methods according to the present invention can be used to treat humans or socially or economically important animal species such as dogs, cats, horses, sheep, cows, goats, or pigs. Methods according to the present invention are not limited to use in humans.

### Pharmaceutical Compositions

In general, a pharmaceutical composition as disclosed herein comprises: (1) a peptide comprising SEQ ID NO: 1 in a physiologically effective quantity; (2) another active agent; and (3) a pharmaceutically acceptable carrier.

The physiologically effective quantity can be determined by one of ordinary skill in the art with reference to the dosages described above.

Conventional pharmaceutically acceptable carriers known in the art can include alcohols, e.g., ethyl alcohol, serum proteins, cholesterol, human serum albumin, liposomes, buffers such as phosphates, water, sterile saline or other salts, electrolytes, glycerol, hydroxymethylcellulose, propylene glycol, polyethylene glycol, polyoxyethylenesorbitan, other surface active agents, vegetable oils, and conventional anti-bacterial or anti-fungal agents, such as parabens, chlorobutanol, phenol, sorbic acid, thimerosal, and the like. A pharmaceutically-acceptable carrier meets industry standards for sterility, isotonicity, stability, and non-pyrogenicity.

The invention is illustrated by the following Examples. These Examples are for illustrative purposes only and are not intended to limit the invention.

### Example 1: Effects of TB₄ and TA₁ on Endothelial Cell Migration

The effects of TB₄ and TA₁ on endothelial cell migration were determined. To investigate the possibility that these peptides effect the migration of normal human vascular endothelial cells (NHVEC) in vitro, a Boyden chamber migration assay was performed.

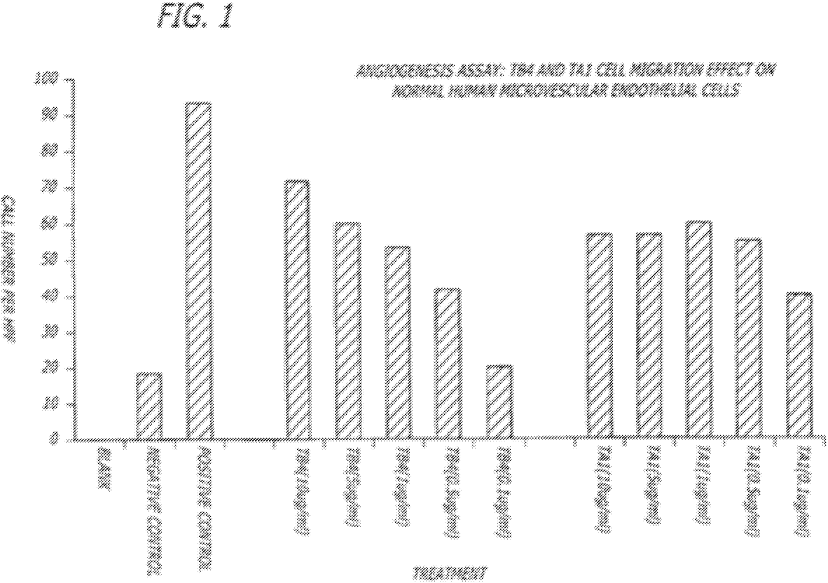

The results are shown in Table 1 and in FIG. 1. In the experiments of Table 1 and FIG. 1, normal human microvesicular endothelial cells are used at 5×10⁴ cells per well. The wells are coated with 100 µg/ml bovine serum albumin Type IV.

These results demonstrate that both TB₄ and TA₁ act as chemoattractants for endothelial cells, stimulating the migration of NHVECs in Boyden chambers. At concentrations of 100 ng/ml, both TB₄ and TA₁ significantly enhanced cell migration over migration in the presence of media alone. Notably, TB₄ and TA₁ also revealed heightened responses at this concentration when compared to three positive controls: vascular endothelial growth factor (VEGF), platelet derived growth factor (PDGF), and fibroblast growth factor (FGF). In addition, endothelial cell migration revealed a dose response to both peptides, as both TB₄ and TA₁ reached a maximal effect on migration at concentrations of 100 ng/ml.

TABLE 1

EFFECT OF PEPTIDES TB₄ AND TA₁ ON CELL MIGRATION
Cell Number per HPF

| Treatment | Concentration | Chamber 1 | Chamber 2 | Chamber 3 | Mean | SD |
|---|---|---|---|---|---|---|
| Blank | Medium only | 0 | 0 | 0 | 0 | 0 |
| Negative | Medium only | 12 | 30 | 13 | 18 | 10 |
| Control | | | | | | |
| Positive | VEGF 10 | 88 | 95 | 97 | 93 | 5 |
| Control | ng/ml | | | | | |
| TB₄ | 10 µg/ml | 70 | 77 | 66 | 71 | 6 |
| TB₄ | 5 µg/ml | 55 | 65 | 57 | 59 | 5 |
| TB₄ | 1 µg/ml | 45 | 47 | 63 | 52 | 10 |
| TB₄ | 0.5 µg/ml | 33 | 39 | 50 | 41 | 9 |
| TB₄ | 0.1 µg/ml | 18 | 12 | 26 | 19 | 7 |
| TA₁ | 10 µg/ml | 51 | 55 | 66 | 57 | 8 |
| TA₁ | 5 µg/ml | 35 | 64 | 72 | 57 | 19 |
| TA₁ | 1 µg/ml | 64 | 69 | 43 | 59 | 14 |
| TA₁ | 0.5 µg/ml | 52 | 53 | 57 | 54 | 3 |
| TA₁ | 0.1 µg/ml | 38 | 45 | 33 | 39 | 6 |

### Example 2: Effects of TB₄ and TA₁ on Endothelial Cell Proliferation

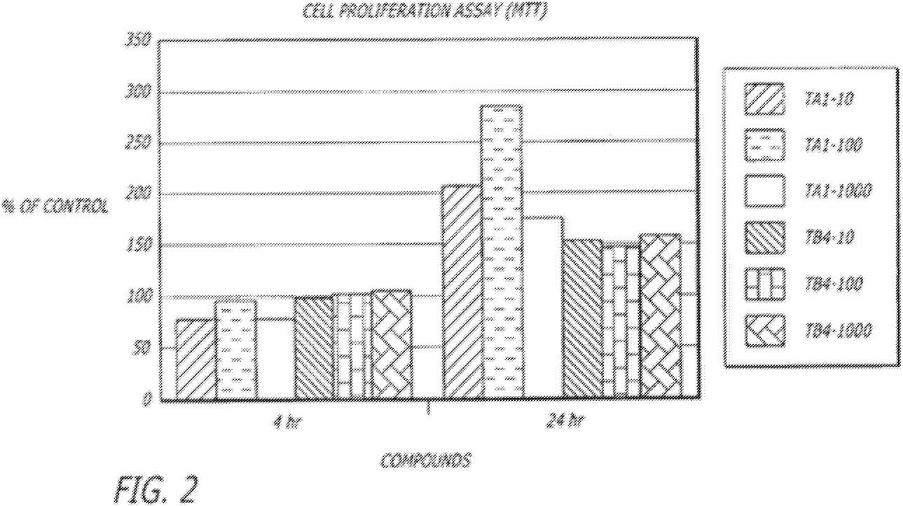

The effects of TB₄ and TA₁ on endothelial cell proliferation were determined using a MTT (tetrazolium) assay. The results are shown in FIG. 2 for 4 hours and 24 hours and for three concentrations of these peptides, 10 ng/ml, 100 ng/ml, and 1000 ng/ml. These results show that these peptides stimulate endothelial cell proliferation. At a concentration of 10 ng/ml, TB₄ and TA₁ maximally stimulated cell proliferation approximately 2-fold and 3-fold over that of the control, respectively.

### Example 3: Hair and Scalp Preparations

This Example describes primary ingredients for hair and scalp preparations. Typically, deionized water is the main carrier for the ingredients.

TABLE 2

Hair and scalp preparation ingredients

| Ingredients | Concentration (grams) |
|---|---|
| Peptide (SEQ ID NO: 1) | 0.00005 - 0.0005 |
| Caprylyl glycol (1,2-octanediol) | 1 - 5 |
| Colloidal sulfur | 0 - 3 |
| Azadirachta indica (neem seed) extract | 0 - 2 |
| Eclipta prostrata (false daisy) extract | 0 - 2 |

### Example 4: Hair Growth Stimulating/Rejuvenation Cream

This Example describes a hair growth stimulating/rejuvenation cream comprising the ingredients shown in Table 3, and results of using the cream versus placebo to increase hair density.

TABLE 3

Hair growth stimulating/rejuvenation cream ingredients

| No. | Ingredient | Concentration (grams) |
|---|---|---|
| 1 | Deionized water | 1 - 2 |
| 2 | Algae extract | 1 - 2 |
| 3 | Glycerin | 1 - 2 |
| 4 | Hydrolyzed milk protein | 1 - 2 |
| 5 | Silane-diol salicylate | 1 - 2 |
| 6 | Butylene glycol | 1 - 2 |
| 7 | Propylene glycol | 1 - 2 |
| 8 | Sulfured amino acid | 1 - 2 |
| 9 | Triethanolamine | 1 - 2 |
| 10 | Sulfo mucopolysaccharide extract | 1 - 2 |
| 11 | Celastrus paniculata extract | 1 - 2 |
| 12 | Colloidal sulfur | 1 - 3 |
| 13 | Trichomonas japonica maxim | 1 - 2 |
| 14 | Spondias amara | 1 - 2 |
| 15 | Rubus parvifolius | 1 - 2 |
| 16 | Artemisia annua | 1 - 2 |
| 17 | Adiantum extract | 1 - 2 |
| 18 | Azadirachta indica extract | 1 - 2 |
| 19 | Solanum tuberosum extract | 1 - 2 |
| 20 | Eclipta prostrata extract | 1 - 2 |
| 21 | Lawsonia inermis extract | 1 - 2 |
| 22 | Homotaurine | 1 - 2 |
| 23 | Barosma betulina | 1 - 2 |
| 24 | Phenoxyethanol | 1 - 2 |
| 25 | Caprylyl glycol (1,2-octanediol) | 1 - 5 |
| 26 | Ethylhexylglycerin | 1 - 2 |
| 27 | Hexylene glycol | 1 - 2 |
| 28 | Sodium benzoate | 1 - 2 |
| 29 | Peptide act-5 (L-K-E-K-K) | 0.00005 - 0.0005 |
| 30 | Acetyl tetrapeptide-3 (Formula 1) | 1 - 2 |
| 31 | Trifolium pretense flower extract (red clover extract) | 1 - 2 |
| 32 | Dextran | 1 - 2 |
| 34 | Potassium sorbate | 0 - 2 |

### Animal Model Study

Materials and methods:
Pigmented C57/BL6 mice, preselected for their telogen I Phase of hair growth were used. The extracts with serum were applied topically to assess telogen to anagen transition.
Immuno histochemical investigations were performed to identify antigens specifically.
Animals in anagen Phase of hair growth were positive for FGF-7.
Animals in telogen phase were positive only for BMP4 Antigen.

Result:
The formulation of Table 3 enhanced the induction of anagen growth in the dorsal skin of mice, characterized by the appearance of inner root sheath along the hair shaft.
The formulation of Table 3 increased FGF-7 while decreasing FGF-5 in C57 and BL6 mice.

Conclusion:
The results suggest that the formulation of Table 3 has the potential to enhance the growth of hair follicles, promoting hair growth through regulation of FGF-7 and FGF-5.
Up regulated FGF-7
Down regulated FGF-5

### Human Clinical Study

A study showing the effect of combining the ingredients in Table 3 was carried out. 30 volunteers with a receding hairline were randomly recruited and divided into two groups.

Pilot Study
1. Group I received placebo (15 subjects)
2. Group II received product (15 subjects)

30 volunteers with a receding hair line were selected
10 male
20 female

Duration: Study conducted for a period of 4 months, daily topical application of the product.

After daily topical application of the product, the scalp was assessed using a digital analysis device that measures hair density and the proportion of hairs in the anagen and telogen phase.

### Observation

Four months after application of placebo and the product, Group II showed the density of hairs (increased hair growth) in anagen phase increased by 13% on average and the hair fall in the telogen phase decreased by 29% relative to control measurements taken at the start of the study.

Group I showed the density of hairs (increased hair growth) in the anagen phase increased by 2% on average and hair fall in the telogen phase increased by 28% relative to control measurements taken at the start of the study.

The ratio of hairs at the hair line in the angen to telogen phase A/T phase increased by 42% in the second group. The results differed greatly from those of group I where the ratio of hairs in the anogen to telogen phases decreased over the course of the experiment.

### Conclusion

Group II experienced the positive effects on hair growth from the test product.

### Example 5: Antibacterial Shampoo

This Example describes an antibacterial shampoo comprising the ingredients shown in Table 4, and results of studies using the shampoo.

TABLE 4

Antibacterial shampoo ingredients

| No. | Ingredient | Concentration (grams) |
|---|---|---|
| 1 | Deionized water | 1 - 2 |
| 2 | TEA laurel sulfate | 1 - 2 |
| 3 | Lauramide DEA | 1 - 2 |
| 4 | Cocomidopropyl betaine | 1 - 2 |
| 5 | Hydrolyzed wheat protein | 1 - 2 |
| 6 | Panthenol | 1 - 2 |
| 7 | Dimethicone | 1 - 2 |
| 8 | Aloe barbadenis (aloe) leaf juice | 1 - 2 |
| 9 | Azadirachta indica (neem seed) oil | 1 - 2 |
| 10 | Oenothera biennis (evening primrose) extract | 1 - 2 |
| 11 | Eclipta prostrata (false daisy) extract | 1 - 2 |
| 12 | Simmondsia (jojoba seed) oil | 1 - 2 |
| 13 | Methylchloroisothiazolinone | 1 - 2 |
| 14 | Methylisothiazolinone | 1 - 2 |
| 15 | Phenoxyethanol | 1 - 2 |
| 16 | Caprylyl glycol (1,2-octanediol) | 1 - 5 |
| 17 | Ethylhexylglycerin | 1 - 2 |
| 18 | Hexylene glycol | 1 - 2 |
| 19 | Fragrance | 1 - 2 |
| 20 | Act-5 peptide (L-K-E-K-K) | 0.00005 - 0.0005 |
| 21 | Colloidal sulfur | 1 - 3 |
| 22 | Acetyl tetrapeptide-3 (Formula 1) | 0 - 2 |
| 23 | Red clove extract | 0 - 2 |

### Animal Model Study

Materials and methods:
Pigmented C57/BL6 mice, preselected for their telogen I Phase of hair growth were used. The extracts with serum were applied topically to assess telogen to anagen transition.
Immuno histochemical investigations were performed to identify antigens specifically.
Animals in anagen Phase of hair growth were positive for FGF-7.
Animals in telogen phase were positive only for BMP4 Antigen.

Result:
The formulation of Table 4 enhanced the induction of anagen growth in the dorsal skin of mice, characterized by the appearance of inner root sheath along the hair shaft.
The formulation of Table 4 increased FGF-7 while decreasing FGF-5 in C57 and BL6 mice.

Conclusion:
The results suggest that the formulation of Table 4 has the potential to enhance the growth of hair follicles, promoting hair growth through regulation of FGF-7 and FGF-5.
Up regulated FGF-7
Down regulated FGF-5

### STATEMENTS REGARDING INCORPORATION BY REFERENCE AND VARIATIONS

All references cited throughout this application, for example patent documents including issued or granted patents or equivalents; patent application publications; and non-patent literature documents or other source material; are hereby incorporated by reference herein in their entireties, as though individually incorporated by reference.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments, exemplary embodiments and optional features, modification and variation of the concepts herein disclosed can be resorted to by those skilled in the art, and that such modifications and variations are considered to be within the scope of this invention as defined by the appended claims. The specific embodiments provided herein are examples of useful embodiments of the invention and it will be apparent to one skilled in the art that the invention can be carried out using a large number of variations of the devices, device components, and method steps set forth in the present description. As will be apparent to one of skill in the art, methods and devices useful for the present methods and devices can include a large number of optional composition and processing elements and steps.

When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure.

It must be noted that as used herein and in the appended claims, the singular forms “a”, “an”, and “the” include plural reference unless the context clearly dictates otherwise. Thus, for example, reference to “a peptide” includes a plurality of such peptides and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described. Nothing herein is to be construed as an admission that the invention is not entitled to antedate such disclosure by virtue of prior invention.

Whenever a range is given in the specification, for example, a range of integers, a temperature range, a time range, a composition range, or concentration range, all intermediate ranges and subranges, as well as all individual values included in the ranges given are intended to be included in the disclosure. As used herein, ranges specifically include the values provided as endpoint values of the range. As used herein, ranges specifically include all the integer values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous and can be used interchangeably with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” can be replaced with either of the other two terms. The invention illustratively described herein suitably can be practiced in the absence of any element or elements, limitation or limitations which is/are not specifically disclosed herein.

All art-known functional equivalents of materials and methods are intended to be included in this disclosure. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed can be resorted to by those skilled in the art, and that such modifications and variations are considered to be within the scope of this invention as defined by the appended claims.

What is claimed is:

1. A preparation comprising:
a peptide comprising a sequence of L-K-E-K-K (SEQ ID NO: 1); and
caprylyl glycol.

2. The preparation of claim 1, wherein the peptide is present at a concentration between 0.1 mg and 0.5 mg and the caprylyl glycol is present at a concentration between 1 gram and 5 grams.

3. The preparation of claim 1 further comprising colloidal sulfur.

4. The preparation of claim 3, wherein the colloidal sulfur is present at a concentration between 1 gram and 3 grams.

5. The preparation of claim 1 further comprising a sulfured amino acid.

6. The preparation of claim 5, wherein the sulfured amino acid is selected from the group consisting of methionine, cysteine, homocysteine and taurine.

7. The preparation of claim 5, wherein the sulfured amino acid is present at a concentration between 1 mg and 5 mg.

8. The preparation of claim 1 further comprising a sulfo mucopolysaccharide extract.

9. The preparation of claim 8, wherein the sulfo mucopolysaccharide is selected from the group consisting of chondroitin sulfate, dermatan sulfate, keratin sulfate, heparin, heparin sulfate, and hyaluronan (hyaluronic acid).

10. The preparation of claim 8, wherein the sulfo mucopolysaccharide extract is present at a concentration between 0.5 grams and 1 gram.

11. The preparation of claim 1 further comprising trifolium pratense flower extract.

12. The preparation of claim 1 further comprising azadirachta indica extract.

13. The preparation of claim 1 further comprising eclipta prostrata extract.

14. The preparation of claim 1 further comprising a compound of formula (I), 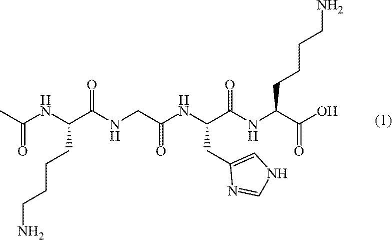 [SMILES: CC(=O)N[C@@H](CCCCN)C(=O)NCC(=O)N[C@@H](Cc1c[nH]cn1)C(=O)N[C@@H](CCCCN)C(=O)O]
.

15. The preparation of claim 1 further comprising one or more carriers, excipients, preservatives and/or diluents.

16. An antibacterial shampoo comprising the preparation of claim 1.

17. A hair growth product comprising the preparation of claim 1.

18. A method of reducing a concentration of bacteria from the hair or scalp of a subject in need thereof, said method comprising administering to said subject a therapeutically effective amount of the preparation of claim 1.

19. A method of inducing hair growth in a subject in need thereof, said method comprising administering to said subject a therapeutically effective amount of the preparation of claim 1.

20. The method of claim 19, wherein the preparation is administered topically.


SEQUENCE LISTING (3 sequences)

SEQ ID NO: 1 - protein, 5 aa, Artificial Sequence
  Synthetic peptide with physiological activity
LKEKK

SEQ ID NO: 2 - protein, 28 aa, Artificial Sequence
  Synthetic peptide with physiological activity
SDAAVDTSSEITTKDLKEKKEVVEEAEN

SEQ ID NO: 3 - protein, 44 aa, Artificial Sequence
  Synthetic peptide with physiological activity
ANKGQAPGEAMKPSFLKEKKEVVERSKEEEGPAKMNLVIEMPKD